(19) United States
(12) Patent Application Publication
(10) Pub. No.: US 2008/0004470 A1
(43) Pub. Date: Jan. 3, 2008

(54) Synthesis of Atomoxetine Hydrochloride

(76) Inventors: Vijayavitthal Thippannachar Mathad, Hyderabad (IN); Mahesh Reddy Ghanta, Hyderabad (IN); Shanmugam Govindan, Hyderabad (IN); Prabhakar Macharla, Hyderabad (IN); Venu Nalivela, Warangal (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/576,106

(22) PCT Filed: Sep. 27, 2005
(86) PCT No.: PCT/US05/34860

§ 371 Date: Mar. 27, 2007

Related U.S. Application Data
(60) Provisional application No. 60/613,363, filed on Sep. 27, 2004.

(51) Int. Cl. C07C 217/48; C07C 213/10

(52) U.S. Cl. 564/347

(57) ABSTRACT

(±)-Atomoxetine oxalate having crystalline Form II and a solid (±)-atomoxetine free base are useful in preparing atomoxetine hydrochloride.

The invention relates to a new crystalline form of N-methyl-3-phenyl-3-(o-tolyloxy) propylamine oxalate (hereinafter referred as “atmoxetine oxalate”) and to an isolation technique for (±)-atmoxetine free base in a solid form, an intermediate useful in the synthesis of atomoxetine hydrochloride.

The compound (−)-N-methyl-3-(2-methylphenoxy)-3-phenylpropylamine, or (−)-N-methyl-3-phenyl-3-(o-tolyloxy)-propylamine hydrochloride, is usually known by its adopted name “atomoxetine hydrochloride.” It is represented as shown in Formula 1 and is a selective norepinephrine reuptake inhibitor. A commercial atomoxetine hydrochloride product is sold as STRATTERA™ in the form of capsules containing 10, 18, 25, 40, 60, 80, or 100 mg of atomoxetine, for treating attention-deficit/hyperactivity disorder.

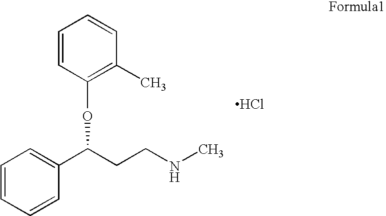

U.S. Pat. No. 4,314,081 describes 3-Aryloxy-3-phenyl polyamines, which possess central nervous system activity. Atomoxetine is a member of the above class of compounds, and is a useful drug for the treatment of depression. Atomoxetine was claimed in U.S. Pat. No. 4,314,081 and the patent describes a process for the preparation of atomoxetine and related compounds in two different ways as depicted below as Scheme A and Scheme B, respectively.

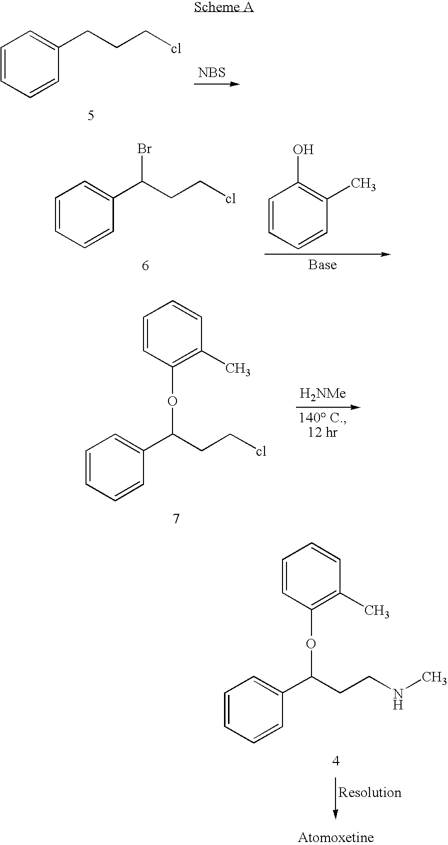

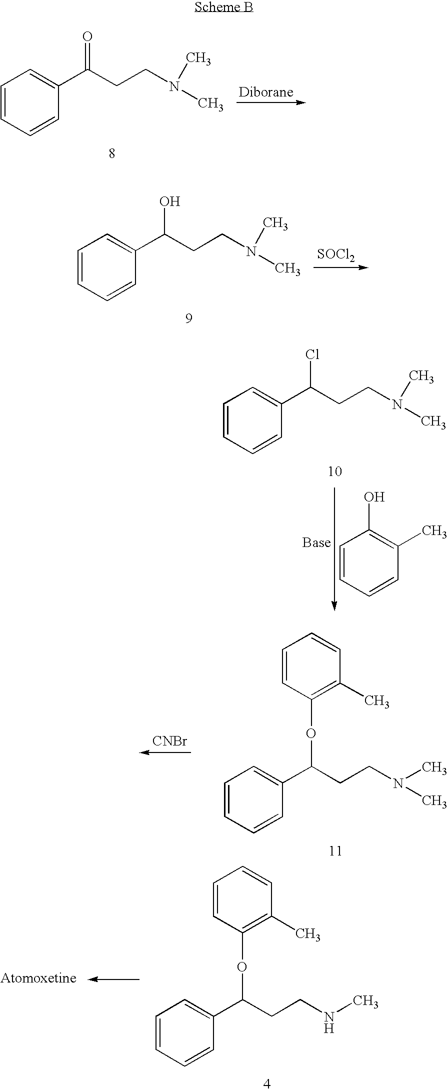

The process illustrated in Scheme A involves the preparation of the atomoxetine using 3-phenyl chloropropyl amine (Formula 5) as a starting material. The process involves bromination of said starting compound (Formula 5) by using N-bromosuccinimide. Further the bromo derivative is condensed with o-cresol to result in a compound of Formula 7, which is then subjected to amination using methylamine. Though the process looks very simple, it involves the following disadvantages:

i) N-bromosuccinimide being a corrosive and sensitive chemical, its usage demands special care;

ii) the workup of the compound formula 7 involves high vacuum (0.03 torr) distillation at 135-145° C., which is a tedious and cumbersome process to carry out at the plant level; and

iii) the reaction conditions involved in some of the steps are harsh, for example the amination reaction is conducted at 140° C. at pressures of 10 kg/cm² for 12 hours in an autoclave.

All the above points make the process not viable for practicing on a commercial scale. Further, as described in U.S. Pat. No. 4,314,081, the free base compounds exist as high boiling oils, but form white crystalline salts.

On the other hand, Scheme B describes the preparation of atomoxetine using β-dimethylaminopropiophenone produced by a Mannich reaction; which is reduced to the hydroxy derivative having Formula 9 using diborane; further the hydroxy compound (Formula 9) is converted to the corresponding chloro derivative of Formula 10 using dry HCl gas and thionyl chloride and is followed by condensation with o-cresol. The said reaction is carried out in methanol at reflux for a duration of five days to achieve the compound of formula 11 and is followed by demethylation using cyanogen bromide to end up with atomoxetine. As can be clearly understood the process is associated with the following problems:

i) the use of costly reagents such as diborane makes the process uneconomical;

ii) the passage of dry HCl gas followed by thionyl chloride addition is very cumbersome and is not advisable in the plant;

iii) this is a time-consuming process, involving a reaction which requires five days for its completion; and

iv) use of cyanogen bromide, which is highly toxic, is not desirable.

All of the above-quoted drawbacks make the process unfriendly to practice in a production plant as well as to the environment.

Further, M. Srebnik et al., Journal of Organic Chemistry, Vol. 53, pages 2916-2920 (1988); E. Corey et al., Tetrahedron Letters, Vol. 30, pages 5207-5210 (1989); U.S. Pat. No. 4,868,344; Y. Gao et al., Journal of Organic Chemistry, Vol. 53, pages 4081-4084 (1988); J. Deeter et al., Tetrahedron Letters, Vol. 31, pages 7101-7104 (1990); and U.S. Pat. No. 4,950,791 disclose stereospecific methods for the preparation of 3-aryloxy-3-phenylpropylamines; the enantiomers of 3-hydroxy-3-phenylpropylamines are prepared by the stereospecific reduction of the corresponding ketones. The thus obtained (S)-3-hydroxy-3-phenyl propylamines are subjected to condensation with aryl alcohols using the Mitsunobo reaction. As can be seen in Scheme C, the reaction involves two critical steps.

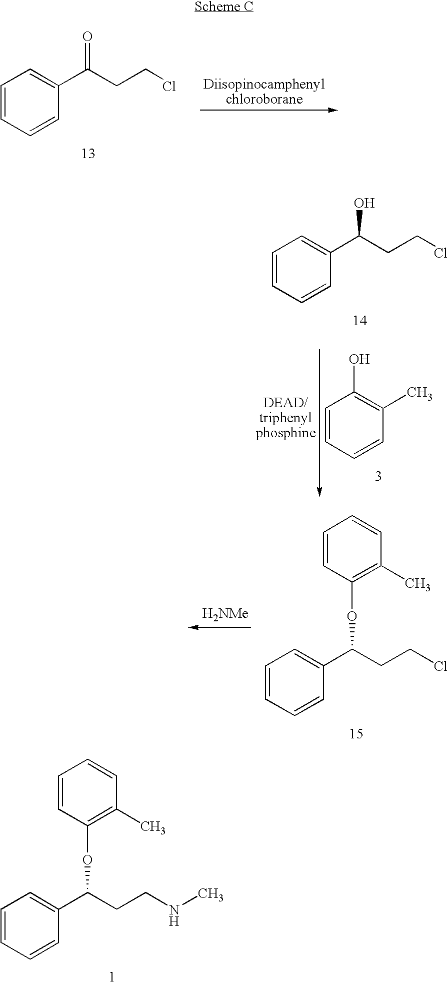

The first critical step is an asymmetric reduction of the ketone to its corresponding alcohol. The second critical step involves the condensation of the obtained enantiomeric alcohol with the corresponding aryl alcohol. The process suffers from the following disadvantages:

1) the reagent used for the asymmetric reduction of the ketone is highly expensive;

2) the reagent diethyl azodicarboxylate (“DEAD”) is expensive;

3) the DEAD reagent is known to be highly carcinogenic, thus creating problems in handling; and

4) the reaction involves the use of triphenylphosphine and DEAD and the resulting byproducts formed in the reaction, phoshineoxide and a hydrazine derivative, are very difficult to remove.

Therefore, commercial applicability of the said process is limited owing to the above noted disadvantages.

International Patent Publication No. WO 00/58262 relates to a stereo-specific process for the preparation of atomoxetine using nucleophilic aromatic displacement of an aromatic ring having a functional group, which can be converted to a methyl group. As can be seen, the process is very lengthy and involves many steps and is thus not commercially desirable.

U.S. Pat. No. 5,847,214 describes the nucleophilic aromatic displacement reaction of 3-hydroxy-3-arylpropylamines with activated aryl halides, for example the reaction of N-methyl-3-phenyl-3-hydroxypropylamine with 4-trifluoromethyl-1-cholro benzene has been reported; the success of this reaction is mainly due to electron withdrawing group on benzene ring of the aryl halides.

U.S. Pat. No. 6,541,668 describes a process for the preparation of atomoxetine and its pharmaceutically acceptable addition salts which comprises reacting an alkoxide of N-methyl-3-phenyl-3-hydroxy propyl amine or an N protected derivative thereof, with 2-fluoro toluene in the presence of 1,3-Dimethyl-2-imidazolidinone (“DMI”) or N-Methyl-3-pyrrolidinone (“NMP”) as the solvent. The process disclosed in the said patent can be shown as Scheme D. Further, the process disclosed in the said patent restricts itself to the solvents DMI and NMP.

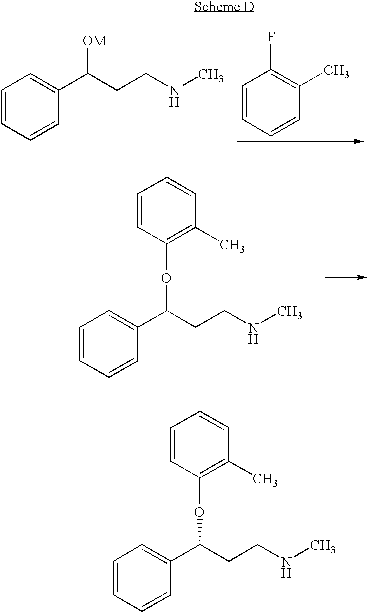

Nevertheless, a new crystalline form of N-methyl-3-phenyl-3-(o-tolyloxy)propylamine oxalate and an isolation technique of (±)-atmoxetine free base in a solid form, an intermediate useful in the synthesis of atomoxetine hydrochloride, is desirable.

### SUMMARY OF THE INVENTION

In an aspect, the invention provides (±)-atomoxetine oxalate having crystalline form II.

Another aspect of the invention provides solid (±)-atomoxetine free base.

An aspect of the invention provides solid (±)-atomoxetine, being prepared by a process comprising hydrolyzing atomoxetine oxalate in an aromatic solvent with a base, removing the solvent to form a residue, mixing the residue with an ester solvent, and isolating solid atomoxetine.

In an additional aspect, the invention provides (±)-atomoxetine oxalate, being prepared by a process comprising reacting (±)-atomoxetine free base with oxalic acid in a ketone solvent and adding an ether solvent.

In a further aspect, the invention provides atomoxetine hydrochloride, prepared by a process comprising:

a) hydrolyzing (±)-atomoxetine oxalate with a base to form atomoxetine;

b) reacting the atomoxetine with an enantiomerically pure organic acid to form a salt;

c) hydrolyzing the salt with a base to form enantiomerically pure atomoxetine; and

d) reacting enantiomerically pure atomoxetine with hydrochloric acid.

The invention also provides atomoxetine hydrochloride containing very low concentrations of any one or more of:
N-methyl-3-hydroxy-3-phenyl propylamine;
N-methyl-3-phenoxy-3-phenyl propylamine hydrochloride;
N-methyl-N-[3-(2-methylphenoxy)-3-phenylpropyl]acetamide;
N-methyl-N-(3-hydroxy-3-phenylpropyl)acetamide;
3-Phenyl-3-(o-methylphenoxy)propylamine hydrochloride;
N-methyl-3-phenyl-3-(m-methylphenoxy)propylamine hydrochloride; and/or
N-methyl-3-phenyl-3-(p-methylphenoxy)propylamine hydrochloride.

### BRIEF DESCRIPTION OF THE DRAWINGS

FIG. 1 is an X-ray powder diffraction pattern of N-methyl 3-phenyl-3-(o-tolyloxy)propylamine oxalate, prepared according to U.S. Pat. No. 4,314,081.

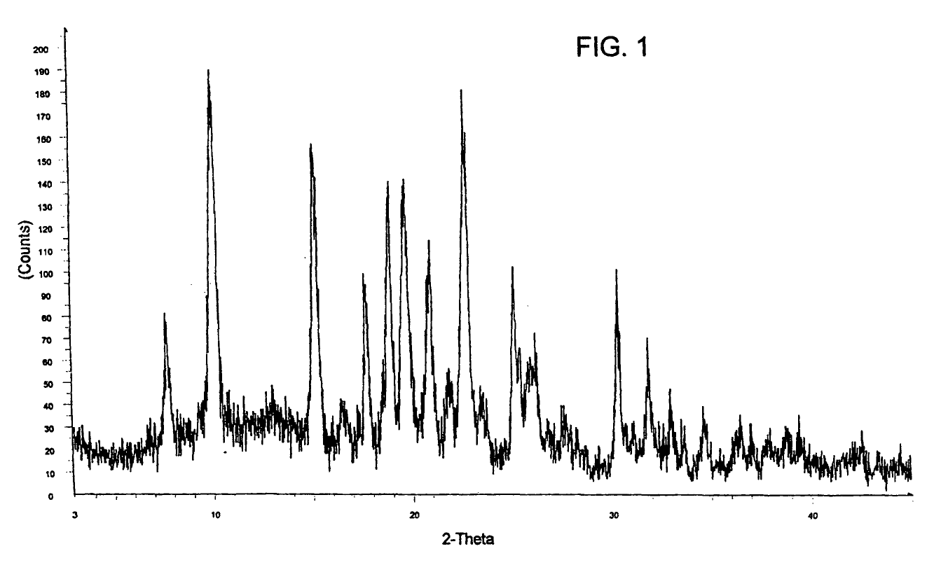

FIG. 2 is an X-Ray powder diffraction pattern of (±)-atomoxetine oxalate Form II, prepared according to Example 2.

FIG. 3 is an infrared absorption spectrum in potassium bromide of (±)-atomoxetine oxalate Form II, prepared according to Example 2.

FIG. 4 is a differential scanning calorimetry analysis of (±)-atomoxetine oxalate Form II according to Example 2.

FIG. 5 is an X-Ray powder diffraction pattern of (±)-atomoxetine free base, prepared according to Example 4.

FIG. 6 is an infrared absorption spectrum in potassium bromide of (±)-atomoxetine free base, prepared according to Example 4.

FIG. 7 is a differential scanning calorimetry analysis of (±)-atomoxetine free base, prepared according to Example 4.

### DETAILED DESCRIPTION

In one aspect, the invention provides a new crystalline form of (±)-atmoxetine oxalate and a process for the preparation thereof. The new crystalline form of (±)-atomoxetine oxalate of the present invention is hereinafter referred to as “Form II.”

The invention relates to a crystalline Form II of (±)-atmoxetine oxalate and to an isolation technique for (±)-atmoxetine free base in a solid form, and to intermediates useful in the synthesis of atomoxetine hydrochloride.

An aspect of the invention, therefore, is crystalline Form II of (±)-atomoxetine oxalate and the process for preparation thereof.

All of the X-ray powder diffraction (“XRPD”) patterns described herein were produced using a Bruker Axe, DS Advance X-ray powder diffractometer with a Cu K alpha-1 radiation source. X-ray powder diffraction patterns are commonly used to identify particular crystalline forms of chemical substances, and an arrangement of peaks is characteristic of a particular crystalline form. However, the peak heights can vary between samples, due to sample preparation differences, and differences between individual diffractometers can result in slight changes to the numerical values associated with peak locations, so an identification should be based primarily upon the relative arrangements of the peaks in a pattern.

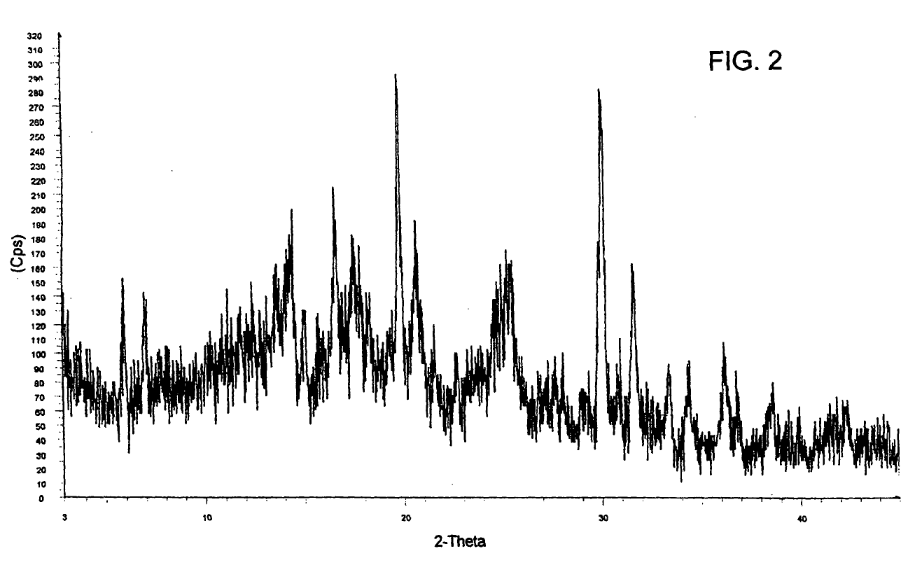

Crystalline Form II of (±)-atmoxetine oxalate is characterized by an XRPD pattern substantially in accordance with FIG. 2. The crystalline Form II of (±)-atomoxetine oxalate is also characterized by an XRPD pattern comprising peaks at about 5.9, 6.9, 19.8, 20.6, 30.1, and 31.6±0.2 degrees 2θ.

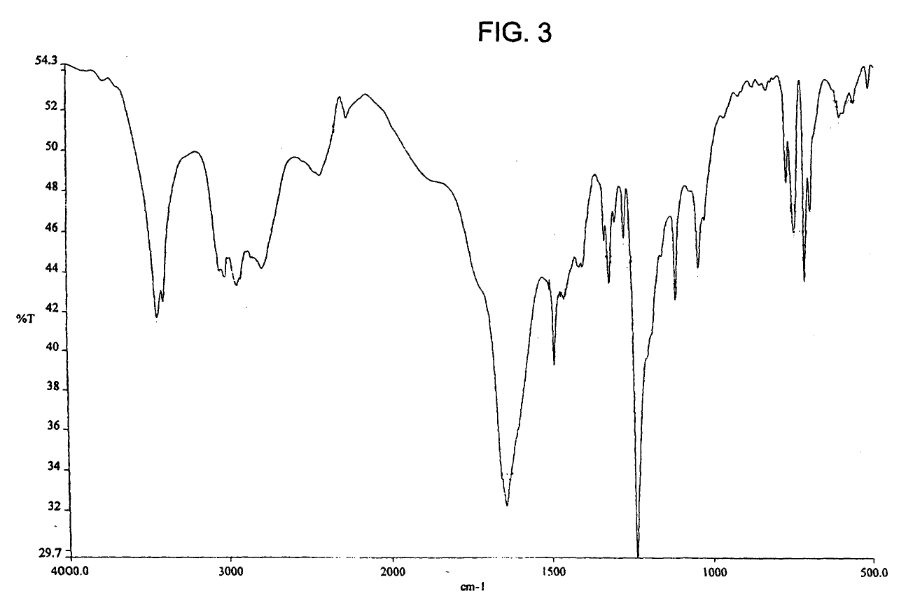

Crystalline Form II of (±)-atmoxetine oxalate is characterized by an infrared absorption spectrum in potassium bromide substantially in accordance with FIG. 3. The crystalline Form II of (±) -atomoxetine oxalate is also characterized by an infrared absorption spectrum in potassium bromide comprising peaks at about 3447, 1493, 1643, 1250, 1120, and 720±5 cm⁻¹.

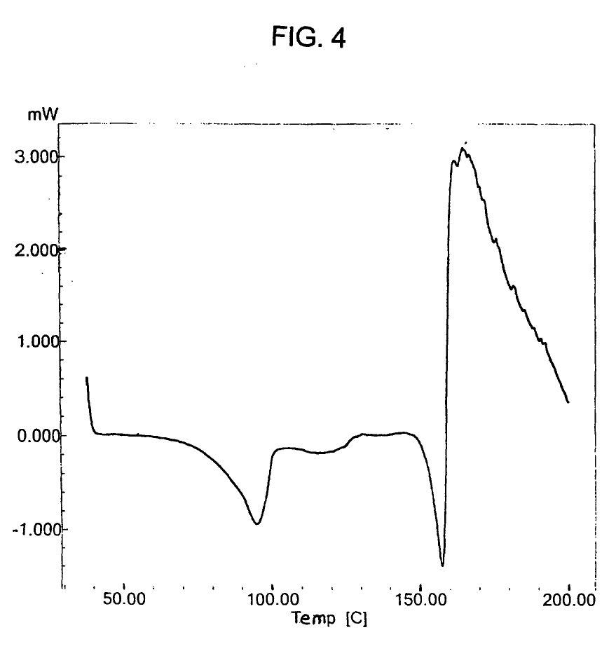

Crystalline Form II of (±)-atmoxetine oxalate is characterized by a differential scanning calorimetry curve substantially in accordance with FIG. 4.

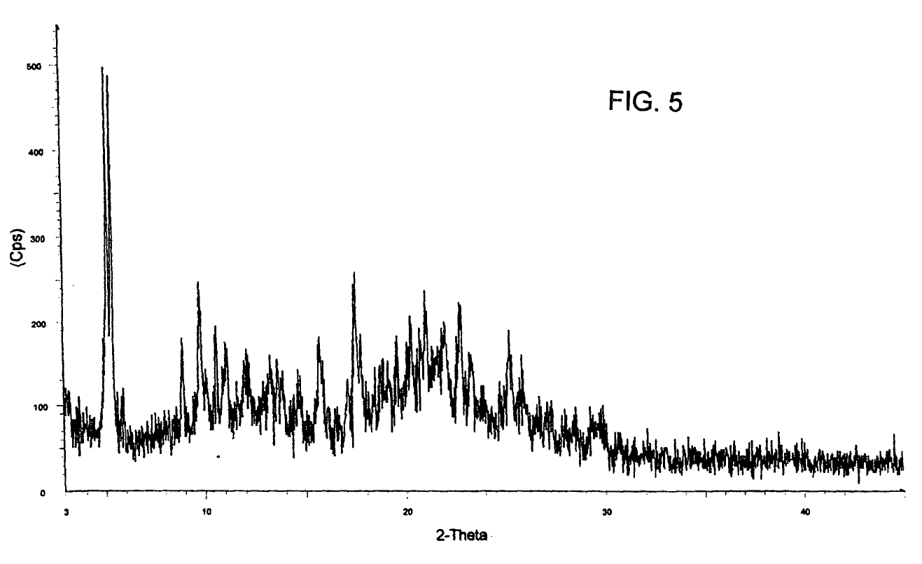

In another aspect, the invention provides (±)-atomoxetine free base in solid form. The solid (±) -atomoxetine free base is characterized by an XRPD pattern substantially in accordance with FIG. 5. The solid (±)-atomoxetine free base is also characterized by an XRPD pattern comprising peaks at about 5.1, 5.3, 9.7, 15.7, 17.4, and 22.8±0.2 degrees 2θ.

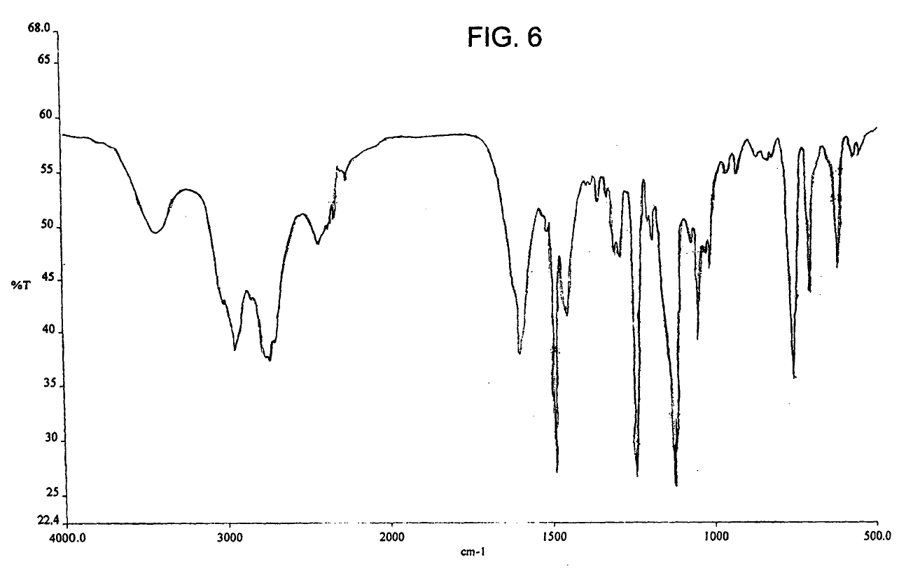

The solid (±)-atomoxetine free base is characterized by an infrared absorption spectrum substantially in accordance with FIG. 6. The solid (±)-atomoxetine free base also is characterized by an infrared absorption spectrum in potassium bromide comprising peaks at about 2742, 1600, 1493, 1241, 1120, and 755±5 cm⁻¹.

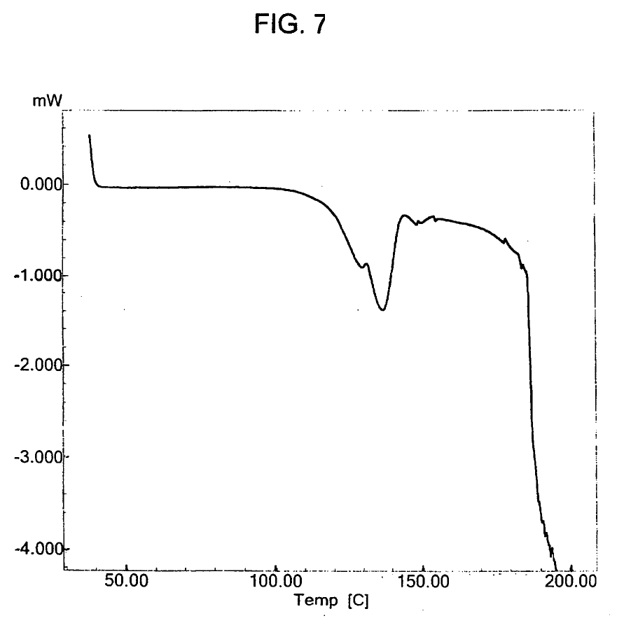

The solid (±)-atomoxetine free base is characterized by a differential scanning calorimetry curve substantially in accordance with FIG. 7.

In a further aspect, the invention provides an isolation technique for (±)-atomoxetine free base in a solid form.

In an embodiment, the invention provides a process for the preparing a crystalline Form II of (±)-atmoxetine oxalate comprising reacting (±)-atomoxetine free base with oxalic acid in a ketonic solvent accompanied by addition of an ether solvent and isolating a solid by filtration to afford the crystalline Form II of (±)-atmoxetine oxalate.

In an embodiment, the process for the preparation of crystalline Form II of (±)-atomoxetine oxalate comprises suspending N-methyl-3-phenyl-3-hyroxypropyl amine, potassium t-butoxide, and 2-fluorotoluene in a polar solvent followed by heating to about 75-150° C., or 120° C. to 130° C., with stirring until the reaction is complete, such as for about 5-15 hours or about 12 hours. The solvent from the reaction mass can be evaporated, such as under reduced pressure under vacuum, and the obtained residue material can be transferred into an autoclave followed by the addition of an alcoholic solvent and caustic lye with simultaneous stirring and heating to a temperature of about 75-150° C., or about 110° C., to complete the reaction, such as for about 5-15 hours or about 6 hours, followed by evaporating the solvent from the reaction mass at a temperature of about 50 to 80° C., or 65 to 69° C., under reduced pressure. To the obtained reaction mass a protic solvent and an hydrocarbon solvent can be added followed by cooling the mixture to about 0 to 10° C., or 5° C., such as in an ice bath, adjusting the pH of the reaction mass with an inorganic acid to about 8-12, or 8-9. The organic and aqueous layers can be separated and the aqueous layer can be extracted with a hydrocarbon solvent. Organic layers are combined and washed with a protic solvent. Then the organic layer can be separated and evaporated under reduced pressure followed by cooling the residue to a temperature of about 15-45° C., or about 30° C. A ketonic solvent can be added to the above residue followed by the addition of an inorganic or organic acid and stirring with simultaneous cooling to about 0-25° C., or about 15° C., then adding an ether solvent followed by stirring; the separated solid can be filtered followed by washing with an ether solvent. The obtained solid can be dried at a temperature of about 35° C.-75° C., or about 50° C., to afford the desired crystalline Form-II of (±)-atomoxetine oxalate.

The solvents that can be used to prepare the crystalline Form II of (±)-atomoxetine oxalate can be chosen depending upon the reaction conditions. Examples of useful polar solvents include, but are not limited to, N,N-dimethyl acetamide, dimethyl formamide, hexamthylphosphoramide, acetonitrile, and the like; alcoholic solvents including, but not limited to, methanol, ethanol, n-propanol, n-butanol, and isopropanol; hydrocarbon solvents including, but not limited to, benzene, toluene, xylene and the like; ketonic solvents including, but not limited to, acetone, methylisobutylketone, t-butyl ketone, and the like; ether solvents including, but not limited to, diethyl ether, dimethyl ether, ethylmethyl ether, methylisobutyl ether, methyl t-butyl ether, and the like. Mixtures of solvents from the different classes are also useful in the invention.

The inorganic or organic acids that are used to prepare the crystalline Form II of (±)-atomoxetine oxalate include, but are not limited to, hydrochloric acid, sulfuric acid, oxalic acid, maleic acid, tartaric acid, hydrobromic acid, methanesulfonic acid, p-toluene sulfonic acid, phosphoric acid, succinic acid, citric acid, and the like.

In one embodiment, (±)-atomoxetine free base can be solid or liquid, and optionally will be isolated.

The crystalline Form II of (±)-atomoxetine oxalate obtained according to the above process can be used for the preparation of atomoxetine hydrochloride.

The crystalline Form II of (±)-atomoxetine oxalate frequently has a purity greater than about 99 area-% by high performance liquid chromatography (“HPLC”).

The crystalline Form II of (±)-atomoxetine oxalate is a free flowing, non solvated stable solid and the process of the present invention is simple, non hazardous, safe to handle, and well suited for commercial production.

In another embodiment, the invention includes an isolation technique for solid (±)-atomoxetine free base comprising the steps of:

a) mixing (±)-atomoxetine oxalate with an aromatic solvent and adding water;

b) adjusting the pH of the suspension to 11-12 by the addition of a base;

c) extracting the aqueous layer with an aromatic solvent;

d) washing the combined organic layers with water;

e) removing solvent to afford the crude solid form;

f) isolating the solid by filtration in an ester solvent; and

g) drying the isolated solid to afford the solid racemic atomoxetine free base.

Useful aromatic solvents include, but are not limited to, toluene, benzene, and xylene.

Useful ester solvents include, but are not limited to, ethyl acetate, isobutyl acetate, and the like.

The solvent can be removed by methods such as distillation, spray drying, rotational evaporation (such as using a Buchi Rotavapor), agitated thin film drying, spin-flash drying, fluid-bed drying, lyophilization, or other techniques that will be apparent to those skilled in the art.

The resultant solid obtained can be further dried by using techniques such as fluid bed drying, spin flash drying, aerial drying, oven drying, suction drying or other techniques known in the art, with or without application of vacuum and/or under inert conditions.

In one embodiment, the resultant solid is dried at a temperature of about 35° C. to 75° C., or 50-55° C., under vacuum. Drying can require a period of as long as about 5 hours to afford the desired racemic atomoxetine free base in solid form.

The racemic atomoxetine free base in solid form obtained as in the above process can be used for the preparation of atomoxetine hydrochloride.

In an embodiment, the present invention relates to a process for preparing atomoxetine and its pharmaceutically acceptable addition salts comprising the following steps:

i. reacting N-methyl-3-phenyl-3-hydroxy propylamine represented by the following formula 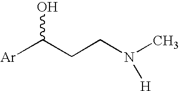
wherein Ar is phenyl, and the pharmaceutically acceptable salts thereof such as the hydrochloride, hydrobromide, etc., with 2-fluorotoluene in the presence of N—N-dimethylacetamide or hexamethylphosphorous triamide as a solvent;

ii. stirring the contents of the reaction mass in step i at temperatures in the range of about 80-140° C., or 110-130° C., until the reaction is complete, such as for about 7-20 hours;

iii. removing the solvent by distillation under vacuum at temperatures less than about 120° C.;

iv. adding an alcohol, such as methanol, and sodium hydroxide and heating to a temperature about 80-140° C., or about 100-110° C.;

v. removing the solvent by distillation under vacuum at temperatures below about 80° C.;

vi. adding water and toluene;

vii. cooling the reaction mass in step vi to about 0-5° C.;

viii. adjusting the pH to a value between about 8 and 9 with an acid, such as dilute hydrochloride acid;

ix. separating the aqueous and the organic layers from the reaction mass of step viii;

x. washing the organic layer of step ix with water;

xi. subjecting the organic layer of step x to evaporation under reduced pressure to remove solvent;

xii. purifying the residue from step xi by forming a salt with an acid such as an inorganic acid such as hydrochloric acid, hydrobromic acid, hydroiodic acid, phosphoric acid, nitric acid, and the like, or an organic acid such as glutaric acid, lactic acid, citric acid, malic acid, fumaric acid, oxalic acid, and the like;

xiii. subjecting the salt formed in step xii to hydrolysis in the presence of a base and dissolving the free base in an organic solvent, followed resolution of the compound by reacting with an optically pure compound such as mandelic acid to form a salt;

xiv. hydrolyzing the salt of the optically pure compound from step xiii with a base to form atomoxetine; and

xv. converting atomoxetine freebase formed in step xiv to the corresponding acid addition salt and isolating a pure enantiomeric salt compound in a suitable solvent, such as isopropyl alcohol.

It frequently is not necessary to isolate the atomoxetine free base. The salt formation of step xv can be performed in situ by adding the desired salt forming acid.

The following reaction Scheme 1 summarizes the process of an embodiment of the invention.

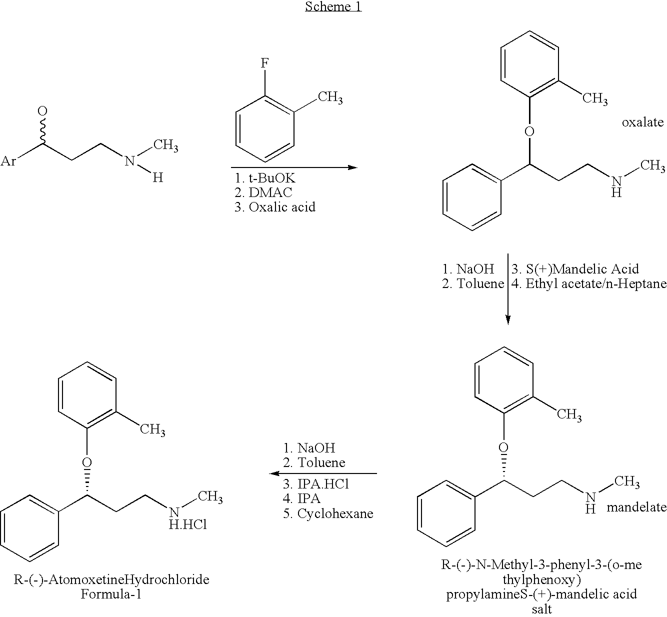

Reaction of the amine with the substituted toluene is facilitated by a prior alkoxide formation at the hydroxyl group of the amine. This can be accomplished by adding at least a stoichiometric equivalent amount of an alkali metal hydride, hydroxide, or alkoxide, or a mixture thereof, to the amine in a solvent, prior to the addition of the substituted toluene. It is not necessary to isolate the formed alkoxide. Representative alkali metal compounds that can be used include, without limitation thereto, sodium or potassium hydride, sodium or potassium hydroxide, and sodium or potassium propoxide or butoxide. Potassium t-butoxide is used in the following examples, but it can readily be replaced by any of numerous other compounds, as will be appreciated by those skilled in the art.

Atomoxetine hydrochloride prepared according to this embodiment has a low level of impurities, as determined by HPLC. For example, it contains less than about 0.15 area-%, or about 0.003 area-%, of N-methyl-3-hydroxy-3-phenyl propylamine of formula (VIII).

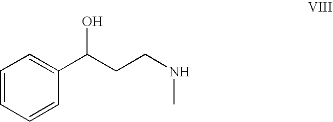

The atomoxetine hydrochloride contains less than about 0.15%, or about 0.009 area-%, or about 0.0008 area-%, of N-methyl-3-phenoxy-3-phenyl propylamine hydrochloride of formula (IX).

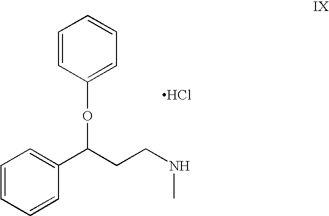

The atomoxetine hydrochloride contains less than about 0.15 area-%, or about 0.03 area-%, of N-methyl-N-[3-(2-methylphenoxy)-3-phenylpropyl]acetamide of formula (X).

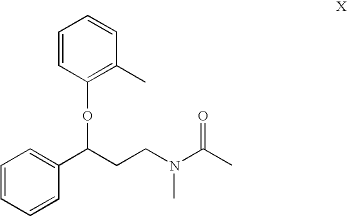

The atomoxetine hydrochloride contains less than about 0.15 area-%, or about 0.001 area-%, of N-methyl-N-(3-hydroxy-3-phenylpropyl)acetamide of formula (XI).

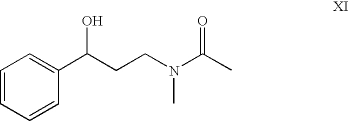

The atomoxetine hydrochloride contains less than about 0.15 area-%, or about 0.003 area-%, of 3-phenyl-3-(o-methylphenoxy)propylamine hydrochloride of formula (XII).

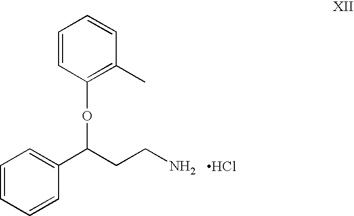

The atomoxetine hydrochloride contains less than about 0.15 area-%, or about 0.06 area-%, or 0.04 area-%, of N-methyl-3-phenyl-(m-methylphenoxy)propylamine hydrochloride of formula (XIII).

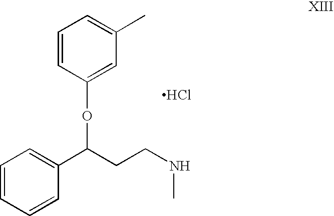

The atomoxetine hydrochloride contains less than about 0.15 area-%, or about 0.02 area-%, or about 0.03 area-%, of N-methyl-3-phenyl-3-(p-methylphenoxy)propylamine hydrochloride of formula (XIV).

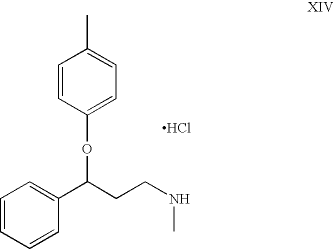

The atomoxetine hydrochloride typically contains less than about 1 area-%, or about 0.5 area-%, or about 0.2 area-%, or about 0.1 area-%, of total impurities.

Atomoxetine hydrochloride obtained according to the present invention is stable at all typical pharmaceutical product storage conditions.

Certain aspects of the process of the present invention will be explained in more detail with reference to the following examples, which are provided by way of illustration only and are not to be construed as limiting the scope of the claimed invention. In the examples, some products are characterized by their infrared (“IR”) absorption spectrum peaks, nuclear magnetic resonance (“NMR”) spectroscopy data, and mass spectrometer (“MS”) analysis.

### EXAMPLE 1

### Preparation of (±)-(N-Methyl-3-Phenyl-3-(o-Tolyloxy) Propylamine Oxalate)

To a solution of 25 g of N-methyl-3-phenyl-3-hydroxyphenyl propylamine in N,N-dimethylacetamide and 19 g. of potassium t-butoxide at 50-60° C. was charged 50 g of 2-fluorotoluene, then the mixture was heated to 120-130° C. and maintained over a period of about 12 to 14 hours followed by distillation of the reaction mass under vacuum at a temperature below 120° C. The reaction mass was cooled to 40 to 50° C., 500 ml of methanol and 300 ml of a 45-50% by weight aqueous sodium hydroxide solution were added, and the mixture was heated in an autoclave to a temperature of about 90 to 120° C., or 100-110° C., for 5-20 hours or 6-7 hours. After the completion of the reaction, as monitored by thin layer chromatography (“TLC”), the solvent was distilled at a temperature below 80° C., 250 ml of water followed by 250 ml of dichloromethane were charged to the residue and the mixture was stirred for 10-15 minutes. The organic layer was separated from the aqueous layer, and the aqueous layer was extracted with 100 ml of dichloromethane. The combined organic layers were subjected to distillation to obtain a thick residue. The obtained residue was further purified by preparing its oxalate salt: the crude residue was charged into acetone (240 ml) followed by the addition of 12.5 g oxalic acid, petroleum ether (240 ml) was charged, and the mixture was stirred for 1-5 hours at 0-5° C. Filtering the obtained solid and washing with a mixture of 100 ml of acetone and petroleum ether in a 1:1 ratio resulted in the recovery of (±)-atomoxetine oxalate Form II with a yield of 55% and HPLC purity of 98.35 area-%.

### EXAMPLE 2

### Preparation of (±)-(N-Methyl-3-Phenyl-3-(o-Tolyloxy) Propylamine Oxalate)-Crystalline Atomoxetine Oxalate Form II

250 milliliters of N,N-dimethylacetamide was taken into round bottom flask to which 115.3 grams of potassium t-butoxide, 100 grams of N-Methyl-3-phenyl-3-(o-tolyloxy) propylamine and 100 grams of 2-fluorotoulene were added, followed by heating to 120 to 130° C. for about 12 to 14 hours. Solvent was distilled from the above reaction mass under vacuum below 118 to 122° C., followed by the addition of 500 milliliters of methanol and 300 milliliters of a 45-50% by weight aqueous solution of sodium hydroxide. The reaction mass was transferred into an autoclave and then heated to a temperature of 105 to 110° C. for about 6 to 7 hours. The solvent was totally distilled off under vacuum at a temperature below 80° C., then 1 liter of water and 1 liter of toluene were added, followed by cooling to 0 to 5° C. and adjusting the pH to a value between 8 and 9 with dilute hydrochloric acid. The above reaction mass was stirred for about 15 to 30 minutes, the organic and aqueous layers were separated, and the aqueous layer was extracted with 400 milliliters of toluene followed by stirring for about 5 to 10 minutes. Organic and aqueous layers were separated and the combined organic layers were washed with 400 milliliters of water. The organic layer was taken into a round bottom flask followed by complete distillation under vacuum below 80° C. and then cooled to 25 to 35° C. 1.3 liters of acetone and 64.2 grams of oxalic acid dihydrate were added to the above reaction mass followed by stirring for about 45 to 60 minutes. The solid mass that was obtained was filtered and washed with 520 milliliters of acetone. The wet solid was transferred into a round bottom flask followed by the addition of 700 milliliters of acetonitrile and stirring for about 30 to 45 minutes. The resulting slurry was filtered and washed with 200 milliliters of acetonitrile followed by drying at 50 to 60° C. for 4 to 5 hours under vacuum to afford crystalline (±)-atomoxetine oxalate having Form II.

### EXAMPLE 3

### Preparation of (±)-(N-Methyl-3-Phenyl-3-(o-Tolyloxy) Propylamine Oxalate)

To a solution of 25 g N-methyl-3-phenyl-3-hydroxyphenyl propylamine in hexamethylphosphorous triamide and 19 g potassium t-butoxide at 50-60° C. was charged 50 g of 2-fluorotoluene and the mixture was heated to 105-110° C. The reaction was maintained for 19-20 hours. After the completion of the reaction (monitored by TLC) there were charged 250 ml of water followed by 250 ml toluene and the mixture was stirred for 10-15 minutes. The aqueous and organic layers were separated, then the aqueous layer was extracted with (2×75 ml) toluene. The combined organic layer was washed with (3×75 ml) water and then subjected to distillation to obtain a thick residue. The residue was dissolved in 150 ml of acetone followed by adding of 12.5 g oxalic acid and 200 ml of isopropyl ether and the mixture was stirred for 1-1.5 hours at 0-5° C., then the obtained solid was separated by filtration and washed with isopropyl ether (100 ml) resulting in the (±)-atomoxetine oxalate with yield 62.4% and a purity of 95.4 area-% by HPLC.

### EXAMPLE 4

### Process for the Preparation of Solid Racemic Atomoxetine Free Base

20 g of racemic atomoxetine oxalate obtained according to any of the above three examples was suspended in a mixture of 400 ml water and toluene followed by adjusting the pH of the suspension to 11-12 with sodium hydroxide, and the stirring was continued for about 10-30 minutes. Organic and aqueous layers were separated followed by extraction of the aqueous layer with (2×40 ml) of toluene and both the organic layers were combined and washed with 80 ml of water. Solvent was distilled under reduced pressure at 55-60° C. The residue was taken into a glass tray an held for about 24 hours at ambient temperature, then was slurried in 100 ml of ethyl acetate followed by filtration, and the obtained solid was dried at a temperature of about 60° C. for 3 hours to afford solid (±)-atomoxetine.

### EXAMPLE 5

### Preparation of R-(−)-N-Methyl-3-Phenyl-3-(o-Tolyloxy) Propylamine-S-(+)-Mandelic Acid Salt

Atomoxetine oxalate (35 grams), dichloromethane (350 ml), water (350 ml), and sodium hydroxide (17.5 grams) were mixed and stirred for 10 minutes. The aqueous layer was separated and extracted with dichloromethane (100 ml). Combined organic layer was washed with 5% sodium hydroxide solution followed by water (100 ml). The organic solvent was removed by distillation under reduced pressure. To the residue was added ethyl acetate (50 ml) followed by L-(+)-mandelic acid (2.9 grams) and the mixture was heated to 50-55° C. for about one hour. Petroleum ether (50 ml) was added and the mixture was cooled to 30-35° C. The separated solid was isolated by filtration and washed with petroleum ether (30 ml), then dried at 50-55° C. under reduced pressure to get the mandelic acid salt of atomoxetine.

### EXAMPLE 6

### Preparation of R-(−)-N-Methyl-3-Phenyl-3-(o-Tolyloxy) Propylamine-S-(+)-Mandelic Acid Salt

500 milliliters of demineralized water, 50 grams of N-Methyl-3-phenyl-3-(o-tolyloxy)propylamine oxalate from Example 2 and 500 milliliters of toluene were taken into a round bottom flask and the pH of the reaction mixture was adjusted to a value between 11.5 to 12.5 by the addition of sodium hydroxide solution, followed by stirring for about 10 to 15 minutes. The reaction mixture was heated to a temperature of 50 to 60° C. for 10 to 15 minutes followed by extraction of the reaction mass with toluene, and then the organic layer was washed with 200 milliliters of demineralized water. The solvent in the organic layer was completely distilled off and the residue was taken into a round bottom flask along with the addition of 11 grams of L-(+)-mandelic acid and 175 grams of ethyl acetate. The above reaction mixture was stirred for 5 to 10 minutes at a temperature of 25 to 35° C. and the reaction mass was checked for precipitation, then 315 milliliters of n-heptane were added followed by cooling the reaction mass to 0 to 5° C. and then stirring for about 45 to 60 minutes at 0 to 5° C. The solid was filtered and washed with a mixture of 75 milliliters of ethyl acetate and n-heptane in the ratio of 1:2. The material that was obtained from filtration was taken into a round bottom flask along with 125 milliliters of isopropyl alcohol followed by heating at a temperature of 75 to 80° C. until a clear solution was obtained. The solution was kept at 75 to 80° C. for about 15 to 30 minutes and then cooled to 0 to 5° C. for about 45 to 60 minutes for crystallization. The solid was separated by filtration and washed with 55 milliliters of isopropyl alcohol, followed by drying at 50 to 55° C. for about 4 to 5 hours under vacuum to get the mandelic acid salt of atomoxetine.

### EXAMPLE 7

### Preparation of Atomoxetine Hydrochloride

The mandelic acid salt of atomoxetine (5.5 grams) from either of Examples 5 or 6, dichloromethane (50 ml), and water (50 ml) were mixed and stirred for 10 minutes at 25-35° C. About 12 ml of a 5% sodium hydroxide solution was added to produce a pH about 10-11 and the mixture was stirred for 10 minutes at 25-35° C. The aqueous and organic layers were separated and the aqueous layer was extracted with dichloromethane (25 ml). The combined organic layer was washed with 5% sodium hydroxide solution (25 ml) followed by washing with water (25 ml). The organic layer was separated and solvent was removed by distillation under reduced pressure. The residue was dissolved in methyl t-butyl ether and isopropyl alcohol was added, then the mixture was stirred for 15 minutes. Hydrochloric acid was added to the solution with stirring and stirring was continued for one hour. Solid was separated and washed with methyl tertiary butyl ether, then dried at 50-55° C. to get the final atomoxetine hydrochloride product. Yield 73.5%, purity 99.5 area-% by HPLC.

### EXAMPLE 8

### Preparation of Atomoxetine Hydrochloride

250 milliliters of water, 25 grams of R-(−)-N-Methyl-3-phenyl-3-(o-tolyloxy) propylamine-S-(+)-mandelic acid salt, and 250 milliliters of toluene were taken in a round bottom flask and cooled to 10 to 15° C. followed by adjusting the pH to a value between 10.5 and 11.5 with sodium hydroxide solution. The reaction mixture was stirred for about 10 to 15 minutes, the organic and aqueous layers were separated followed by the extraction of the aqueous layer with 100 milliliters of toluene and the organic layer was washed with 100 milliliters of water. The solvent was distilled off completely under vacuum at a temperature below 60 to 65° C. and the residue was taken into a round bottom flask along with 50 milliliters of isopropyl alcohol, followed by cooling to 0 to 5° C. An equal volume of an 18% by weight solution of hydrochloric acid in isopropyl alcohol was added slowly over about 30 to 45 minutes to the mixture at a temperature of 0 to 5° C. with stirring, then the mixture was refluxed for about 30 to 45 minutes until a clear solution was obtained. The above solution was cooled to 0 to 5° C. to produce a precipitate, and 145 milliliters of cyclohexane were added and the mixture was maintained for about 45 to 60 minutes at a temperature of 0 to 5° C. Solids were removed by filtration and washed with 500 milliliters of cyclohexane, and then taken into a round bottom flask to which 50 milliliters of isopropyl alcohol was added, and then refluxed at temperatures of 70 to 75° C. for about 30 to 45 minutes and cooled to 0 to 5° C. for crystallization. The crystals were filtered and washed with 25 milliliters of isopropyl alcohol followed by drying at 45 to 50° C. for about 6 to 8 hours under vacuum to get atomoxetine hydrochloride. Atomoxetine hydrochloride thus obtained had a purity of 99.35 area-% by HPLC.

The crude atomoxetine was taken into round bottom flask to which 50 ml of isopropyl alcohol was added, and then refluxed at temperature of about 70 to 75° C. over a period of 30 to 45 minutes and cooled to 0 to 5° C. for crystallization. The crystals were filtered and washed with 25 ml of isopropyl alcohol followed by drying at 45 to 50° C. for about 6 to 8 hours under vacuum to get atomoxetine hydrochloride. Atomoxetine hydrochloride thus obtained has a purity of 99.9 area-% by HPLC with a yield of 70.4%.

### EXAMPLE 9

### Preparation of (±)-N-Methyl-3-Hydroxy-3-Phenylpropylamine

1200 liters of water were placed into a reactor along with 400 kg of N-Methyl-N-benzyl-2-benzoyl ethamine hydrochloride. 19.2 kg of 5% PdCl₂ on carbon was suspended in 40 liters of water and the solution was set aside. 1200 liters of ethyl acetate were added to the mixture containing water and N-Methyl-N-benzyl-2-benzoyl ethamine hydrochloride, followed by the addition of the above-prepared 5% PdCl₂ suspension along with 50 liters of water under a nitrogen atmosphere. Hydrogen pressure of about 3.0 to 3.5 kg/cm² was applied to the reaction mass at a temperature of about 55 to 57° C. Hydrogen pressure was removed and the reaction mass was maintained for about 1 hour and than cooled to a temperature of about 35 to 37° C. under a nitrogen atmosphere of about 0.5 to 1.0 kg/cm². The reaction mass was filtered through a leaf filter and transferred into another reactor, stirred for about 10 to 15 minutes and allowed to settle for about 20 to 30 minutes. The aqueous layer was separated and extracted with 400 liters of toluene by stirring the mixture and then allowing the layers to separate, then the aqueous layer was removed. 172 kg of a 45-50% by weight aqueous solution of sodium hydroxide were added to the organic layer and stirred for 15 to 20 minutes, the layers were allowed to settle, the aqueous layer was separated, and the sodium hydroxide treatment was repeated. The organic layer was transferred into a reactor and heated to a temperature of about 75 to 90° C., allowed to settle for about 15 to 30 minutes and the water was removed. The reaction mass was heated to reflux and the water removed from the reaction mass azeotropically under reflux below 120° C. The solvent was distilled completely under vacuum below 100° C. for about 1 hour, and finally at a temperature of about 80 to 85° C. to produce the product.

Melting Range: 50-60° C.

IR: 3067, 3030 (Ar—CH), 2965, 2946 (Ali-CH), 1483 (Ar C═C bending), and 1235 (Ar C—O—C) cm⁻¹.

1H NMR: (DMSO-d6, 200 MHz): δ 7.39 (m, 1H), δ 7.27 (m, 1H), δ 7.22 (m, 1H), δ 7.25 (m, 1H), δ 7.32 (m, 1H J=8.4), δ 4.94 (dd, 1H) δ 1.8 (m, 2H), 2.8 (m, 2H), δ 2.4 (s, 3H).

13C NMR: (DMSO-d6, 200 MHz): δ 127, 127, 125.8, 125.8, 126.4, 71.7, 48.9, 36.8, 36.0.

MS: m/z 166 (100%, M+1).

### EXAMPLE 10

### Preparation of N-Methyl-3-Phenoxy-3-Phenylpropylamine Hydrochloride

100.0 g of N-methyl-3-phenyl-3-hydroxy propyl amine, 87 g of fluorbenzene, 115.3 g of potassium tertiary butoxide, and 250 ml of dimethyl acetamide were taken into a round bottom flask and heated to a temperature of about 120° C. with stirring for about 11 to 12 hours, heating was stopped and the stirring was continued over a period of about 10 hours. The above reaction mass was transferred into round bottom flask along with the addition of 500 ml of methanol and 300 ml of a 45-50% by weight aqueous solution of sodium hydroxide and subjected to stirring with simultaneous heating to a temperature of about 110° C., then was cooled to a temperature of about 32° C., and the reaction mass was taken into another round bottom flask and subjected to distillation to a temperature of about 65° C. followed by the addition of 1000 ml of water to the contents in the flask and cooling to a temperature of about 0 to 5° C. pH of the reaction mass was adjusted with 300 ml of 5% aq. hydrochloric acid and 1000 ml of toluene were added with stirring. The aqueous and organic layers were separated and the aqueous layer was extracted with 1000 ml of toluene. Both organic layers were combined and washed with 800 ml of water. The organic layer was subjected to distillation to a temperature of about 65° C. and to the residue 568 ml of acetone were added followed by stirring and cooling to a temperature of about 5° C., and formed solid was filtered and washed with acetone. The solid mass was suction dried for about 10 minutes and finally dried at a temperature about 50° C.

Melting Range: 165 to 170° C.

IR: 3067 (Ar—CH), 2846 (Ali-CH), 1597, 1497 (Ar C═C bending), and 1236 (Ar C—O—C) cm⁻¹.

1H NMR: (DMSO-d6, 200 MHz): δ 7.41 (d, 1H J=8.4), δ 7.34 (m, 1H), δ 7.27 (m, 1H), δ 7.34 (m, 1H), δ 7.40 (d, 1H J=8.4), δ 5.3 (dd, 1H J=4.4, 8.0), δ 6.74 (d, 1H J=7.6), δ 6.97 (t, 1H J=7.6), δ 6.75 (t, 1H J=7.4), δ 7.11 (d, 1H J=7.2), δ 3.0 (m, 2H).

13C NMR: (DMSO-d6, 200 MHz): δ 125.8, 128.5, 126.2, 128.5, 125.8, 140.8, 75.6, 155.0, 112.9, 127.7, 120.2, 130.4, 125.8, 34.1, 45.1, and 32.2.

MS: m/z 242 (100%, M+1)

### EXAMPLE 11

### Preparation of N-Methyl-N-[3-(2-Methylphenoxy)-3-Phenylpropyl]Acetamide

100 g of atomoxetine free base, 1 liter of dichloromethane, and 47.5 g of triethylamine were placed into a round bottom flask which was disposed in an ice bath, followed by the dropwise addition of 36.9 g of acetyl chloride dissolved in 50 ml of dichloromethane with simultaneous stirring at a temperature of about 10° C. over a period of about 2 hours until a solid precipitated, followed by the addition of 500 ml of water with stirring. The organic and aqueous layers were separated and the organic layer was washed with 400 ml of water, then the organic matter was separated and subjected to distillation at a temperature of about 40° C. for period of about 45 minutes.

IR: 3062 (Ar—CH), 2928 (Ali-CH), 1602 (Ar C═C bending), and 1237 (Ar C—O—C) cm⁻¹.

1H NMR: (DMSO-d6, 200 MHz): δ 7.41 (d, 2H J=8.4), δ 7.34 (m, 2H), δ 7.22 (m, 1H), δ 7.10 (m, 1H), δ 6.9 (d, 1H J=8.4), δ 6.7 (d, 1H J=8.4), δ 5.36 (dd, 1H J=4.4, 8.0), δ 6.74 (d, 1H J=7.6), δ 6.97 (t, 1H J=7.6), δ 6.75 (t, 1H J=7.4), δ 7.11 (d, 1H J=7.2) δ 3.4 (m, 2H), δ 2.1 (s, 3H), δ 1.8 (s, 3H).

13C NMR: (DMSO-d6, 200 MHz): δ 125.8, 128.5, 126.2, 128.5, 125.8, 140.8, 75.6, 155.0, 112.9, 127.7, 120.2, 130.4, 125.8, 34.1, 45.1, 32.2, and 16.1.

MS: m/z 298 (100%, M+1)

### EXAMPLE 12

### Preparation of N-Methyl-N-(3-Hydroxy-3-Phenylpropyl) Acetamide

50 g of N-methyl-3-phenyl-3-hydroxy propyl amine and 250 ml of dichloromethane were placed into a round bottom flask followed along with 50.5 ml of triethylamine and the mixture was cooled to a temperature of about 0 to 5° C., then 260 ml of acetyl chloride was added slowly in a dropwise manner for a period of about 35 minutes and the reaction mass was maintained for about 1 hour at the same temperature. 500 ml of water were added to the reaction mass and the aqueous and organic layers were separated, followed by washing the organic layer with water. The organic layer was separated and subjected to distillation at a temperature of about 45° C. for about 50 minutes. To the residue 275 ml of ethyl acetate were added and the mixture was cooled to a temperature of about 5° C. with stirring. The solid was isolated by filtration and washed with 100 ml of ethyl acetate, subjected to suction drying, and finally dried at a temperature of about 60° C.

Melting Range: 55-62° C.

IR: 3234 (Ar—CH), 295028 (Ali-CH), and 3234 (OH) cm⁻¹.

1H NMR: (DMSO-d6, 200 MHz): δ 7.37 (m, 1H), δ 7.3 (m, 1H), δ 7.2 (m, 1H), δ 7.35 (m, 1H), δ 5.3 (dd, 1H), δ 4.54 (d, OH), δ 3.42 (d, 2H), δ 1.88 (s, 3H), δ 1.8 (s, 2H).

13C NMR: (DMSO-d6, 200 MHz): δ 128, 128, 125.6, 125.6, 126.7, 145.8, 146, 71.7, 46.

MS: m/z 208 (100%, M+).

### EXAMPLE 13

### Preparation of 3-Phenyl-3-(o-Methylphenoxy)Propylamine Hydrochloride

20 g of 3-chloro-1-(o-methoxyphenyl)propyl benzene, 75 ml of methanol and 66 ml of 15% aqueous ammonia were placed into an autoclave and heated to a temperature of about 100° C. with stirring for a period of about 14 hours. After cooling the autoclave, the reaction mass was removed and subjected to distillation at a temperature of about 60° C. followed by the addition of 250 ml of water and 400 ml of diisopropyl ether to the residue. The mixture was stirred for about 10 minutes and the aqueous layer and organic layer were separated. The aqueous layer was extracted with 100 ml of diisopropyl ether and the organic layers were combined and washed with 200 ml of water, then the organic layer was separated and subjected to distillation at a temperature of about 60° C. The obtained free base was transferred into a round bottom flask along with the addition of 50 ml of isopropyl alcohol. The reaction mass was stirred and cooled to 0-5° C. followed by the slow addition of 27 ml of 10% hydrochloric acid in isopropanol with stirring at a temperature of about 5° C., over about 1 hour, then the mixture was heated to a temperature of about 70 to 75° C. with stirring. The reaction mass was cooled to a temperature of about 0 to 5° C., and 150 ml of cyclohexane were added with stirring. The obtained solid was filtered, followed by washing with 100 ml of cyclohexane, subjected to suction drying for about 25 minutes and finally dried at a temperature of about 50° C.

Melting Range: 142 to 144° C.

IR: 2965 (Ali-CH), 1590, 1602 (Ar C═C bending), and 1241 (Ar C—O—C) cm⁻¹.

1H NMR: (DMSO-d6, 200 MHz): δ 7.41 (d, 1H J=8.4), δ 7.34 (m, 1H), δ 7.27 (m, 1H), δ 7.34 (m, 1H), δ 7.40 (d, 1H J=8.4), δ 5.35 (m, 1H J=4.4, 8.0), δ 6.74 (d, 1H J=7.6), δ 6.97 (t, 1H J=7.6), δ 6.75 (t, 1H J=7.4), δ 7.11 (d, 1H J=7.2) δ 2.33 (s, 3H), δ 3.2 (m, 2H), δ 2.4 (s, 2H).

13C NMR:(DMSO-d6, 200 MHz): δ 130.6, 128.7, 128.7, 127.8, 126.4, 125.6, 125.6, 120.6, 112.8, 126.8, 155.1, 140, 76.6, 36.1, and 16.6.

MS: m/z 242 (100%, M+1).

### EXAMPLE 14

### Preparation of N-Methyl-3-Phenyl-(m-Methylphenoxy)Propylamine Hydrochloride

50 g of atomoxetine oxalate and 500 ml of water were taken into a round bottom flask and stirred, followed by the dropwise addition of 10 ml of a 45-50% by weigh aqueous sodium hydroxide solution, and then 500 ml of toluene were added with stirring. The aqueous and organic layers were separated followed by extracting the aqueous layer with 400 ml of toluene, and the toluene was separated and washed with 500 ml of water. The organic layer was separated and subjected to distillation at a temperature of about 60° C. for about 1 hr and the obtained free base was transferred into a round bottom flask along with the addition of 100 ml of isopropyl alcohol. The reaction mass was subjected to stirring and cooled to 0-5° C. followed by the slow addition of 47 ml of isopropanol containing 10% hydrochloric acid with simultaneous stirring at a temperature of about 5° C. for about 1 hour, then the mixture was heated to a temperature of about 70 to 75° C. with stirring. The reaction mass was cooled to a temperature of about 0 to 5° C., and 300 ml of cyclohexane were added with stirring. The obtained solid was filtered, followed by washing with 400 ml of cyclohexane, and subjected to suction drying. 84 ml of isopropyl alcohol were added to the wet solid and the mixture was stirred with simultaneous heating to a temperature of about 70° C., followed by cooling the to a temperature of about 0 to 5° C. The formed solid was filtered and washed with 28 ml of isopropyl alcohol followed by suction drying for about 10 minutes and the solid was finally dried at a temperature of about 50° C.

Melting Range: 122 to 127° C.

IR: 2957, 2867 (Ali-CH), 1513 (Ar C═C bending), and 1237 (Ar C—O—C) cm⁻¹.

1H NMR: (DMSO-d6, 200 MHz): δ 7.41 (m, 1H), δ 7.36 (m, 1H), δ 7.2 (m, 1H), δ 7.38 (m, 1H), δ 7.40 (m, 1H), δ 5.5 (m, 1H), δ 7.02 (d, 1H), δ 6.7 (d, 1H), δ 6.5 (d, 1H), δ 6.57 (s, 1H), δ 2.3 (d, 1H), δ 2.9 (m, 1H), δ 9.26 (s, NH), δ 2.5 (s, 1H), δ 2.15 (s, 1H).

13C NMR: (DMSO-d6, 200 MHz): δ 126.0, 129.13, 127.8, 128.6, 129.13, 145, 76.0, 157, 112, 121, 126.0, 138.8, 34.2, 46.0, and 32.4, 21.

MS: m/z 256 (100%, M+1).

### EXAMPLE 15

### Preparation of N-Methyl-3-Phenyl-3-(p-Methylphenoxy)Propylamine Hydrochloride

60 g of atomoxetine oxalate and 600 ml of water were placed into a round bottom flask and stirred. 12 ml of a 45-50% by weight aqueous sodium hydroxide solution and 600 ml of toluene were added with stirring. Organic and aqueous layers were separated and the aqueous layer was extracted with 480 ml of toluene, then separated organic layers were combined and subjected to distillation at a temperature of about 60° C. 137 ml of isopropyl alcohol was added to the residue and the mixture was stirred, followed by cooling to a temperature of about 0 to 5° C., and 47 ml of isopropanol containing 10% hydrochloric acid were added slowly. The mixture was heated to a temperature of about 75° C. followed by cooling to 0 to 5° C. 450 ml of cyclohexane were added with stirring and the obtained solid was filtered, washed with 120 ml of cyclohexane and subjected to suction drying for about 15 to 30 minutes. The obtained 40 g of wet solid was transferred into a round bottom flask, 137 ml of isopropyl alcohol were added, and the mixture was heated to a temperature of about 75° C. with simultaneous stirring. The reaction mass was cooled to 0 to 5° C. and the formed solid was filtered and washed with 68 ml of isopropyl alcohol, followed by suction drying for about 10 minutes, and finally the solid was dried at a temperature of about 50° C.

Melting Range: 122-127° C.

IR: 3019 (Ar—CH), 2945 (Ali-CH), 1488 (Ar C═C bending), and 1257 (Ar C—O—C) cm⁻¹.

1H NMR: (DMSO-d6, 200 MHz): δ 7.41 (m, 1H), δ 7.36 (m, 1H), δ 7.2 (m, 1H), δ 7.38 (m, 1H), δ 7.40 (m, 1H), δ 5.51 (m, 1H), δ 6.991 (d, 1H), δ 6.78 (d, 1H), δ 6.77 (d, 1H), δ 6.79 (d, 1H), δ 2.3 (d, 1H), δ 2.9 (m, 1H), δ 9.25 (s, NH), δ 2.49 (s, 1H), δ 2.15 (s, 1H).

13C NMR: (DMSO-d6, 200 MHz): δ 126.9, 129.7, 128.1, 128.62, 127.7, 140, 76.2, 155.1, 125.6, 115.9, 126.0, 34.2, 45.2, 32.3, and 20.0.

MS: m/z 256 (100%, M+1).

1. (±)-Atomoxetine oxalate having crystalline form II.

2. The (±)-atomoxetine oxalate of claim 1, having an X-ray diffraction pattern using Cu Kα-1 radiation substantially in accordance with FIG. 2.

3. The (±)-atomoxetine oxalate of claim 1, having an X-ray diffraction pattern using Kα-1 radiation comprising peaks at about 5.9, 6.9, 19.8, 20.6, 30.1, and 31.6±0.2 degrees 2θ.

4. The (±)-atomoxetine oxalate of claim 1, having an infrared absorption spectrum in potassium bromide substantially in accordance with FIG. 3.

5. The (±)-atomoxetine oxalate of claim 1, having an infrared absorption spectrum in potassium bromide comprising peaks at about 3447, 1643, 1493, 1250, 1120, and 720±5 cm⁻¹.

6. The (±)-atomoxetine oxalate of claim 1, having a differential scanning calorimetry curve substantially in accordance with FIG. 4.

7. The (±)-atomoxetine oxalate of claim 1, being prepared by a process comprising reacting (±)-atomoxetine free base with oxalic acid in a ketone solvent and adding an ether solvent.

8. Atomoxetine hydrochloride, prepared by a process comprising:
a) hydrolyzing the (±)-atomoxetine oxalate of claim 1 with a base to form atomoxetine;
b) reacting the atomoxetine with an enantiomerically pure organic acid to form a salt;
c) hydrolyzing the salt with a base to form enantiomerically pure atomoxetine; and
d) reacting enantiomerically pure atomoxetine with hydrochloric acid.

9. Solid (±)-atomoxetine free base.

10. The solid (±)-atomoxetine of claim 9, having an X-ray diffraction pattern using Cu Kα-1 radiation substantially in accordance with FIG. 5.

11. The solid (±)-atomoxetine of claim 9, having an X-ray diffraction pattern using Cu Kα-1 radiation comprising peaks at about 5.1, 5.3, 9.7, 15.7, 17.4, and 22.8±0.2 degrees 2θ.

12. The solid (±)-atomoxetine of claim 9, having an infrared absorption spectrum in potassium bromide substantially in accordance with FIG. 6.

13. The solid (±)-atomoxetine of claim 9, having an infrared absorption spectrum in potassium bromide comprising peaks at about 2742, 1600, 1493, 1241, 1120, and 755±5 cm⁻¹.

14. The solid (±)-atomoxetine of claim 9, having a differential scanning calorimetry curve substantially in accordance with FIG. 7.

15. The solid (±)-atomoxetine of claim 9, being prepared by a process comprising hydrolyzing atomoxetine oxalate in an aromatic solvent with a base, removing the solvent to form a residue, mixing the residue with an ester solvent, and isolating solid atomoxetine.

16. Atomoxetine hydrochloride containing less than about 0.15 area-%, as determined by high performance liquid chromatography, of N-methyl-3-hydroxy-3-phenyl propylamine.

17. The atomoxetine hydrochloride of claim 16, containing less than about 0.003 area-%, of N-methyl-3-hydroxy-3-phenyl propylamine.

18. Atomoxetine hydrochloride containing less than about 0.15 area-%, as determined by high performance liquid chromatography, of N-methyl-3-phenoxy-3-phenyl propylamine hydrochloride.

19. The atomoxetine hydrochloride of claim 18, containing less than about 0.009 area-% of N-methyl-3-phenoxy-3-phenyl propylamine hydrochloride.

20. The atomoxetine hydrochloride of claim 18, containing less than about 0.0008 area-% of N-methyl-3-phenoxy-3-phenyl propylamine hydrochloride.

21. Atomoxetine hydrochloride containing less than about 0.15 area-%, as determined by high performance liquid chromatography, of N-methyl-N-[3-(2-methylphenoxy)-3-phenylpropyl]acetamide.

22. The atomoxetine hydrochloride of claim 21, containing less than about 0.03 area-% of N-methyl-N-[3-(2-methylphenoxy)-3-phenylpropyl]acetamide.

23. Atomoxetine hydrochloride containing less than about 0.15 area-%, as determined by high performance liquid chromatography, of N-methyl-N-(3-hydroxy-3-phenylpropyl)acetamide.

24. The atomoxetine hydrochloride of claim 23, containing less than about 0.001 area-% of N-methyl-N-(3-hydroxy-3-phenylpropyl)acetamide.

25. Atomoxetine hydrochloride containing less than about 0.15 area-%, as determined by high performance liquid chromatography, of 3-phenyl-3-(o-methylphenoxy)propylamine hydrochloride.

26. The atomoxetine hydrochloride of claim 25, containing less than about 0.003 area-% of 3-phenyl-3-(o-methylphenoxy)propylamine hydrochloride.

27. Atomoxetine hydrochloride containing less than about 0.15 area-%, as determined by high performance liquid chromatography, of N-methyl-3-phenyl-(m-methylphenoxy)propylamine hydrochloride.

28. The atomoxetine hydrochloride of claim 27, containing less than about 0.06 area-% of N-methyl-3-phenyl-(m-methylphenoxy)propylamine hydrochloride.

29. The atomoxetine hydrochloride of claim 27, containing less than about 0.04 area-% of N-methyl-3-phenyl-(m-methylphenoxy)propylamine hydrochloride.

30. Atomoxetine hydrochloride containing less than about 0.15 area-%, as determined by high performance liquid chromatography, of N-methyl-3-phenyl-3-(p-methylphenoxy)propylamine hydrochloride.

31. The atomoxetine hydrochloride of claim 30, containing less than about 0.02 area-% of N-methyl-3-phenyl-3-(p-methylphenoxy)propylamine hydrochloride.

32. The atomoxetine hydrochloride of claim 30, containing less than about 0.03 area-% of N-methyl-3-phenyl-3-(p-methylphenoxy)propylamine hydrochloride.
